(19) United States
(12) Patent Application Publication
(10) Pub. No.: US 2023/0364224 A1
(43) Pub. Date: Nov. 16, 2023

(54) Pharmaceutical Composition For Allergen Specific Immunotherapy

(71) Applicant: HOPITAUX UNIVERSITAIRES DE GENEVE, Geneva (CH)

(72) Inventors: Jean-Christoph CAUBET, Geneve (CH); Francois GRAHAM, Montreal (CA)

(21) Appl. No.: 18/025,985

(22) PCT Filed: Sep. 16, 2021
(86) PCT No.: PCT/EP2021/075498

§ 371 Date: Mar. 13, 2023

(30) Foreign Application Priority Data
Sep. 16, 2020 (EP) 20196418.6

(51) Int. Cl. A61K 39/35; A61K 31/137; A61K 45/06; A61P 37/08

(52) U.S. Cl. CPC A61K 39/35; A61K 31/137; A61K 45/06; A61P 37/08; A61K 2039/577

(57) ABSTRACT

The invention relates to intradermal administration of an allergen for desensitization and/or tolerance induction of allergic patients and for treatment and prophylaxis of allergy. More specifically, it relates to the administration of an allergen and a local vasoconstrictor through the intradermal route to enable a slow release of allergen and lower side effects of therapy.

### FIELD OF THE INVENTION

The invention relates to a composition comprising an allergen for use in a method of desensitization and/or tolerance induction of allergic patients and in a method of treatment and prophylaxis of allergy, wherein the composition is administered through the intradermal route. More specifically, it relates to a composition comprising an allergen and a local vasoconstrictor for use in a method of desensitization and/or tolerance induction of allergic patient and in a method of treatment and prophylaxis of allergy, wherein the composition is administered through the intradermal route, thus enabling a slow release of allergen and reducing the side effects of therapy.

### BACKGROUND OF THE INVENTION

Allergic diseases are a worldwide health epidemic that affect up to 30% of the population. The most common allergies are allergic rhinitis and asthma to inhalant allergens, food allergies, drug allergies, insect allergies, and skin allergies.

Allergen immunotherapy (AIT) involves recurrent administration of doses of the allergen to which the patient is allergic, to diminish allergic symptoms. Desensitization is a temporary phenomenon which lasts as long as the treatment is maintained, while tolerance is acquired when symptoms do not recur after the treatment is stopped.

The mechanism of action of AIT remains unknown, although it is associated with a switch from a Th2 (allergen type 2 helper T cells) to a Th1 (T helper type 1) response, initial rise followed by reduction in specific IgE (immunoglobulin E), and increased IgG4 (Immunoglobulin G4) against the allergen. Allergen-specific IgG4 is thought to inhibit allergen activation of mast cells and basophils, and increase of allergen-specific IgG4 during AIT is generally reflective of clinical efficacy of AIT.

Many different routes have been explored for AIT including subcutaneous (SCIT), sublingual (SLIT), epicutaneous (EPIT), intradermal (IDIT), intralymphatic (ILIT) and intranasal (IN). SCIT is the best studied form of immunotherapy and has been used for over a century to treat allergic rhinoconjunctivitis, allergic asthma, and insect venom hypersensitivity (1). In addition, some data supports effectiveness for the treatment of atopic dermatitis with aeroallergen sensitization. SCIT generally involves subcutaneous injections of allergenic extract on a weekly basis until maintenance dose is reached, and a pursuit of injections on a monthly basis for 3 to 5 years. Rush and ultrarush protocols exist for insect venom hypersensitivity. For inhalant immunotherapy, the response to this therapy is moderate, with a rate of around 30 to 40% improvement of symptoms compared to placebo. Severe systemic reactions can occur after SCIT which from less than 1% to 34% of patients depending on protocol used, with approximately 1 death per 2.5 million injections. SLIT has been used to treat inhalant allergy, with a better safety profile but at the cost of being less effective than SCIT, and compliance can become a problem due to treatment required on a daily basis for a long period of time.

Other novel routes of immunotherapy such as IDIT and ILIT have also been studied for inhalant AIT with promising results (2).

The intradermal route (intracutaneous) has been investigated in the prior art related to desensitization to an allergen as early as 1933 (11). Such method has been implemented mainly for injection of respiratory allergens, particularly pollen extract. Although the author describes this method as providing particularly efficient desensitization, this publication also refers to a previous article (13) describing abandonment of the intradermal method for pollen allergy desensitization, because it caused too many constitutional reactions, and its replacement by subcutaneous therapy accompanied by additional measures to keep the side effects low, i.e use of a tourniquet and admixture of adrenalin and ephedrin with the pollen extract. Therefore, the combined teachings of these documents tend to suggest that increased efficacy of intradermal injection compared to subcutaneous injection is at the cost of safety and that intradermal administration of allergen is not a solution of choice in view of the side effects, and in particular the systemic side effects of such method of AIT. The intradermal route has only been seldomly mentioned in literature since that time. In particular, Ordman et al. (12) reported in 1961 that intradermal administration is used almost exclusively in South Africa, and has even been abandoned there for a long time by specialists, the subcutaneous administration route being preferred for safety reasons. More recently, WO2014/013123A1 described a method for desensitization to a wide variety of allergens, including food allergens, wherein the intradermal route was selected. However, in this method chemically modified allergoids had to be used for the sake of safety of the treatment. Such allergoids had reduced allergenicity compared to natural allergens.

Thus, even though the intradermal route is known since a long time for performing AIT, this route never turned out to be a preferred option and it has never been associated with a safer way of achieving desensitization of allergens in their natural form.

Systemic side effects are especially high in the case of food allergy AIT, significantly higher than in AIT with respiratory allergens, such as pollen. Currently, no treatment is available in routine practice for food allergies, although several types of immunotherapy are under investigation (3). SCIT was studied in the 1990s to treat peanut allergy, but this method was abandoned due to a high rate of systemic reactions (4). Indeed, subcutaneous injections of food allergen are largely contraindicated by allergists since that time. Most recent studies have focused on oral immunotherapy (OIT), with a high rate of clinical desensitization (up to 80%) in randomized controlled trials (RCTs). Although promising, rates of sustained unresponsiveness (SR) (i.e. considered as complete remission) are low in most studies and therapy is limited by adverse reactions and long-term necessity of ingesting foods that patients may develop aversion to over time. Indeed, daily ingestion of foods can lead to systemic reactions in up to 20% of patients requiring emergency treatment, even during maintenance doses. Epicutaneous immunotherapy (EPIT) is a safer alternative, although this comes with much lower efficacy.

Thus, safety is a paramount problem in AIT. Strategies to increase safety of allergenic extracts used in AIT include modified native allergens (allergoids) with less reaginic epitopes, recombinant hypoallergenic proteins, and synthetic peptides that only recognize T and/or B cell epitopes. Although interesting, these strategies have been shown to be expensive, potentially less effective due to the alteration of allergens and removal of important epitopes, and can also lead to sensitization and anaphylaxis to the modified protein/peptide. Native allergens clearly have an advantage in that they more accurately reflect the spectrum of allergenic proteins which lead to sensitization in the first place. Another strategy to improve safety in AIT is to use adjuvant treatments (ex: anti-IgE therapy) to potentially reduce adverse effects. On the other hand, these therapies are very expensive and have not been shown to improve rates of sustained unresponsiveness in the treatment of food allergy.

It is the purpose of the present invention to provide a solution to these problems, by reducing the occurrence of systemic reactions, without compromising the efficiency of the treatment. It is a particular objective of the present invention to provide a method that allows secure desensitization and/or induction of tolerance to a wide range of allergens, including those which tend to cause significant systemic reactions, such as food allergens.

Epinephrine is used on a regular basis with local anesthetics in clinical practice to prolong the duration of anesthesia and limit systemic absorption and toxic reactions (5). Epinephrine combined with local anesthetics have been studied extensively in humans by both the subcutaneous and intradermal routes and are well tolerated as long as maximum recommended doses (MRD) are respected. Epinephrine injected subcutaneously or intradermally has been shown to reduce cutaneous blood flow (6, 7). Epinephrine syringe rinses (ie. coating of the syringe with epinephrine) performed prior to subcutaneous pollen injections have been shown to lower local reactions associated with subcutaneous AIT (8, 9). However, this technique is not standardized, and many factors can affect its reliability (quantity of liquid persisting in the syringe, type of syringe used . . . ). It also has the potential to be harmful due to unknown concentrations of epinephrine injected. Finally, this technique has not been studied using other routes, i.e. the effect of epinephrine intradermally is not expected to be the same as in the subcutaneous tissue.

### BRIEF DESCRIPTION OF THE INVENTION

Performing AIT using a local vasoconstrictor and allergen within a predefined range has surprisingly been shown to be an interesting avenue to treat allergic diseases, by increasing potential therapeutic doses and efficacy, while reducing adverse events, especially for immunotherapy with allergens associated with higher risk of adverse events such as food and cat allergens.

Applicants propose to perform allergen specific immunotherapy in the presence of a local vasoconstrictor, thereby reducing the rate of systemic side effects of AIT. More specifically, the invention relates to administration of an allergen and a local vasoconstrictor via the intradermal route to enable a slow release of allergen and lower side effects of therapy.

In a first aspect, the invention relates to a composition or a set of compositions for use in a method for desensitizing and/or inducing tolerance to an allergen in an allergic patient, for treating allergy in an allergic patient or for the prophylaxis of allergy in a patient, characterized in that said composition or set of compositions comprises a dose of 5 ng to 500 μg of a local vasoconstrictor per administration event, a therapeutic dose of 0.1 ng to 10 mg of the allergen per administration event, and further characterized in that the dose of the local vasoconstrictor is administered by intradermal administration to the patient and in that the therapeutic dose of the allergen is administered by intradermal administration to the patient, simultaneously with or subsequently to the intradermal administration of the local vasoconstrictor, wherein the intradermal administration of the allergen is performed in the area of the dermis where the local vasoconstrictor is administered.

In other words, the invention relates to a method for desensitizing and/or inducing tolerance to an allergen in an allergic patient, for treating allergy in a patient or for the prophylaxis of allergy in a patient, said method comprising intradermal administration of a dose of 5 ng to 500 μg of a local vasoconstrictor per administration event and simultaneous or subsequent intradermal administration of a therapeutic dose of 0.1 ng to 10 mg of the allergen per administration event to the patient, wherein the intradermal administration of the allergen is performed in the area of the dermis where the vasoconstrictor is administered.

In still other words, the invention relates to the use of a composition for the manufacture of a medicament or to the use of a set of compositions for the manufacture of a set of medicaments, for desensitizing and/or inducing tolerance to an allergen in an allergic patient, for treating allergy in an allergic patient or for the prophylaxis of allergy in a patient, characterized in that said composition or set of compositions comprises a dose of 5 ng to 500 μg of a local vasoconstrictor per administration event, and a therapeutic dose of 0.1 ng to 10 mg of the allergen per administration event and further characterized in that the dose of the local vasoconstrictor is administered by intradermal administration to the patient and in that the therapeutic dose of the allergen is administered by intradermal administration to the patient, simultaneously with or subsequently to the intradermal administration of the local vasoconstrictor, wherein the intradermal administration of the allergen is performed in the area of the dermis where the local vasoconstrictor is administered.

In one particular embodiment of the first aspect, the allergen is administered by intradermal injection.

In one particular embodiment of the first aspect, the invention relates to a composition for use in a method for desensitizing and/or inducing tolerance to an allergen in an allergic patient, for treating allergy in a patient or for the prophylaxis of allergy in a patient, wherein said composition is administered by intradermal injection and comprises, per injection, a mixture of a therapeutic dose of 0.1 ng to 10 mg of allergen, between 5 ng to 500 μg of a local vasoconstrictor, and at least one suitable pharmaceutical excipient.

In other words, this specific embodiment of the first aspect of the invention relates to a method for desensitizing and/or inducing tolerance to an allergen in an allergic patient, for treating allergy in a patient or for the prophylaxis of allergy in a patient, said method comprising administering a composition by intradermal injection, said composition comprising, per injection, a mixture of a therapeutic dose of 0.1 ng to 10 mg of allergen, between 5 ng to 500 μg of a local vasoconstrictor, and at least one suitable pharmaceutical excipient.

In still other words, this specific embodiment of the first aspect of the invention relates to the use of a composition for the manufacture of a medicament for desensitizing and/or inducing tolerance to an allergen in an allergic patient, for treating allergy in a patient or for the prophylaxis of allergy in a patient, wherein said composition is administered by intradermal injection and comprises, per injection, a mixture of a therapeutic dose of 0.1 ng to 10 mg of allergen, between 5 ng to 500 μg of a local vasoconstrictor, and at least one suitable pharmaceutical excipient.

Also provided are the following specific embodiments:
an immunotherapeutic composition for use in a method of desensitizing and/or inducing tolerance to an allergen in an allergic patient, wherein said composition is administered by intradermal injection and comprises, per injection: a mixture of a therapeutic dose of 0.1 ng to 10 mg of allergen, between 5 ng to 500 μg of a local vasoconstrictor, and at least one suitable pharmaceutical excipient.
an intradermal desensitization and/or tolerance induction immunotherapeutic composition suitable for injection to an allergic patient, said immunotherapeutic composition comprising per injection: a therapeutic dose of 0.1 ng to 10 mg of allergen, between 5 ng to 500 μg of a local vasoconstrictor, and at least one suitable pharmaceutical excipient; and
an intradermal desensitization and/or tolerance induction immunotherapeutic composition suitable for injection to an allergic patient, said immunotherapeutic composition comprising per injection: a therapeutic dose of 0.1 ng to 10 mg of allergen, between 5 ng to 500 μg of a local vasoconstrictor, and at least one suitable pharmaceutical excipient, for use in the treatment or prophylaxis of allergic diseases.

In a second aspect, the present invention relates to an immunotherapeutic kit for use in a method of desensitizing and/or inducing tolerance to an allergen in an allergic patient, for treating allergy in a patient or for the prophylaxis of allergy in a patient, wherein said kit comprises:
1) a first set of vials with progressive 5 to 10-fold dilutions of a stock allergen, optionally in a suitable pharmaceutical excipient,
2) a second pre-prepared vial with a fixed concentration of local vasoconstrictor, optionally in a suitable pharmaceutical excipient, and optionally a fixed concentration of a local anaesthetic and/or of an adjuvant,
3) a third set of vials for mixing said first vial with said second vial so as to prepare a dose of said allergen,
   characterized in that said dose of said allergen prepared in step 3) is administered by intradermal administration and wherein said dose of said allergen comprises from 0.1 ng to 10 mg of said allergen and from 5 ng to 500 μg of said local vasoconstrictor per administration event.

In other words, the present invention relates to a method of desensitizing and/or inducing tolerance to an allergen in an allergic patient, for treating allergy in a patient or for the prophylaxis of allergy in a patient, comprising:
a) preparing a dose of an allergen wherein said dose of said allergen comprises from 0.1 ng to 10 mg of said allergen and from 5 ng to 500 μg of said local vasoconstrictor per administration event by using a kit comprising:
   1) a first set of vials with progressive 5 to 10-fold dilutions of a stock allergen, optionally in a suitable pharmaceutical excipient,
   2) a second pre-prepared vial with a fixed concentration of local vasoconstrictor, optionally in a suitable pharmaceutical excipient, and optionally a fixed concentration of a local anaesthetic and/or of an adjuvant,
   3) a third set of vials for mixing said first vial with said second vial so as to prepare the dose of said allergen; and
b) administering the dose of the allergen to the patient by intradermal administration.

In still other words, the present invention relates to an immunotherapeutic kit for use in the manufacture of a medicament for desensitizing and/or inducing tolerance to an allergen in an allergic patient, for treating allergy in a patient or for the prophylaxis of allergy in a patient, wherein said kit comprises:
1) a first set of vials with progressive 5 to 10-fold dilutions of a stock allergen, optionally in a suitable pharmaceutical excipient,
2) a second pre-prepared vial with a fixed concentration of a local vasoconstrictor, optionally in a suitable pharmaceutical excipient, and optionally a fixed concentration of a local anaesthetic and/or of an adjuvant,
3) a third set of vials for mixing said first vial with said second vial so as to prepare a dose of said allergen,
   characterized in that said dose of said allergen prepared in step 3) is administered by intradermal administration and wherein said dose of said allergen comprises from 0.1 ng to 10 mg of said allergen and from 5 ng to 500 μg of said local vasoconstrictor per administration event.

In one particular embodiment of the second aspect, the dose of said allergen prepared in step 3) is administered by intradermal injection.

Also provided is an intradermal desensitization and/or tolerance induction immunotherapeutic kit, comprising:
1) a first set of vials with progressive 5 to 10-fold dilutions of a stock allergenic extract in a suitable pharmaceutical excipient,
2) a second pre-prepared vial with a fixed concentration of local vasoconstrictor in a suitable pharmaceutical excipient and optionally a fixed concentration of a local anaesthetic and/or of an adjuvant,
3) a third set of vials for mixing said first vial with said second vial so as to prepare an injected dose of said allergen from 0.1 ng to 10 mg and a dose of said local vasoconstrictor from 5 ng to 500 μg per injection.

Other objects and advantages of the invention will become apparent to those skilled in the art from a review of the ensuing detailed description, which proceeds with reference to the following illustrative drawings, and the attendant claims.

### BRIEF DESCRIPTION OF THE FIGURES

FIG. 1: Intradermal injection of 0.05 mL of fish allergenic extract combined with epinephrine 100 mcg/mL causes skin blanching consistent with local vasoconstriction.

FIG. 2: Thermographic images of intradermal injection of fish allergenic extract with or without epinephrine (100 mcg/mL) on forearm of fish allergic subject were captured using a Flir One Pro thermal imaging camera. Darker areas represent colder temperatures (allergenic extract at room temperature is black compared to surrounding warm skin which is light grey/white). Allergenic extract alone is rapidly absorbed by local blood vessels (dark grey filaments surrounding black papule where intradermal injection occurred) immediately during injection as seen with syringe and needle still present (A) and this continues 30 seconds after injection as seen with dark vessels still present (B). This rapid absorption of allergenic extract is abolished when epinephrine 100 mcg/mL is added to fish allergenic extract both during injection (C) and after 30 seconds (D), as shown by absence of vessels surrounding black papule.

FIG. 3: Fish-allergic patient treated weekly to biweekly with intradermal cod extract without epinephrine, followed by intradermal cod extract with epinephrine after a treatment pause. Cod-specific IgG4 levels increase slowly in the first 5 months of treatment with cod allergic extract without epinephrine (absolute increase of 0.86 mgA/L) (A). Continuing treatment with cod extract in the presence of epinephrine (100 mcg/mL) causes a rapid increase in cod-specific IgG4 after only 1 month of treatment (absolute increase of 1.05 mgA/L) (A). Monthly cod specific IgG4 increase (mgA/L) is significantly lower using cod extract without epinephrine (mean of 0.21 mgA/L per month) when compared to cod extract with epinephrine (1.05 mgA/L in one month) (B).

### DETAILED DESCRIPTION OF THE INVENTION

Although methods and materials similar or equivalent to those described herein can be used in the practice of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the singular forms “a”, “an” and “the” include plural references unless the context clearly dictates otherwise.

The presence of broadening words and phrases such as “one or more,” “at least,” “but not limited to” or other like phrases in some instances shall not be read to mean that the narrower case is intended or required in instances where such broadening phrases may be absent.

Also, the use of “or” means “and/or” unless otherwise stated.

Similarly, “comprise”, “comprises”, “comprising”, “include”, “includes” and “including” are interchangeable and not intended to be limiting. The term “comprise” is generally used in the sense of include, that is to say permitting the presence of one or more features or components.

It is to be further understood that where descriptions of various embodiments use the term “comprising”, those skilled in the art would understand that in some specific instances, an embodiment can be alternatively described using language “consisting essentially” of or “consisting of”.

The terms “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “an effective amount” refers to an amount necessary to obtain a physiological effect. The physiological effect may be achieved by one application dose or by repeated applications. The dosage administered may, of course, vary depending upon known factors, such as the physiological characteristics of the particular composition; the age, health and weight of the subject; the nature and extent of the symptoms; the kind of concurrent treatment; the frequency of treatment; and the effect desired and can be adjusted by a person skilled in the art.

“Pharmaceutically acceptable excipients or carriers” are any materials that do not interfere with the pharmacological activity of the active ingredient(s) or degrade the body functions of the subject to which it can be administered but facilitate fabrication of dosage forms or administration of the composition.

Preferably, an “excipient” is any pharmacologically inert, pharmaceutically acceptable substance or mixture of substances useful for formulating a pharmaceutical composition. Examples of potentially useful excipients include solvents, cosolvents, diluents, surfactants, co-surfactants, thickeners, film-forming agents, stabilisers, antioxidants, solubilisers, pH-adjusting agents, colouring agents, hydrogels, thermogels and the like.

The skin is composed of two primary layers: the outer epidermis and the underlying dermis. The subcutaneous tissue (also called subcutis or hypodermis) lies beneath the skin and is mainly composed of loose connective tissue.

An “intradermal administration” is the administration of a substance in such way that said substance diffuses into the dermis. Intradermal administration can be achieved by intradermal injection, by application of the composition on the skin or with any form of needle-free device using any of the following technologies including but not limited to jet injection, iontophoresis, electroporation, thermal ablation and sonophoresis. In addition, the invention may be administered using any form of microneedle. In the case of application on the skin, the composition can for example be provided as a cream, a paste, a gel, an ointment, a solution, a lotion or an oil, optionally administered in the form of an impregnated patch.

An “intradermal injection” is one specific way of intradermal administration. It is the administration of a substance by injection into the dermal layer of the skin. An intradermal injection is different from a “subcutaneous injection”, anciently referred to as “subcuticular injection”, as such subcutaneous injection refers to the administration of a substance into the subcutaneous tissue, i.e. directly below the dermis and epidermis. Typically, intradermal injections are performed with a hypodermic needle and volume injected varies from 0.02 to 0.1 mL.

The dermis is immunologically active and contains a large amount of Langerhans cells, dermal dendritic cells (DCs) and lymphatics compared to subcutaneous tissue, which is largely composed of adipose tissue and few DCs. In addition, intradermal injection of allergen leads to a 100-fold higher rate of drainage to regional lymph nodes when compared to subcutaneous injections, potentially leading to more efficient pulsing of lymph node DCs (10).

An “allergen” is defined as any substance that can elicit an allergic reaction.

An “allergenic extract” is one specific form in which the allergen can be provided for the purpose of the present invention. It is an injectable sterile solution composed of allergens and other nonallergenic substances. Allergenic extracts are available as aqueous, glycerinated or lyophilized formulations.

Non-Standardized Allergenic Extracts are labelled in weight/volume (w:v) and/or protein nitrogen units (PNU)/millilitre (a measure of total protein), and are generally supplied as sterile aqueous stock concentrates up to 1/10 w:v or 40,000 PNU/millilitre. Weight/volume describes the extraction ratio, or the amount of crude allergen added to the extracting fluid. For example, a 1:10 w:v extract indicates one gram of crude material was used to prepare 10 mL of liquid extract. The amount and composition of extracted materials will also vary depending on the kind of antigen, the extracting fluid, duration of extraction, pH, temperature, and other variables.

As used herein, the dose and/or range of “allergen” (in ng, μg, or mg) in the proposed immunotherapeutic composition refers to the quantity of crude extracted natural allergen, modified natural allergen, synthetic allergen, recombinant allergen, allergoid, peptide or peptide fragment allergens and mixtures or combinations thereof per 0.05 mL intradermal administration.

A “vasoconstrictor” is defined as any agent that can cause vasoconstriction, which is the narrowing of blood vessels caused by contraction of muscular walls of these vessels. In the context of the present invention, the effect of vasoconstriction is to reduce blood flow to the skin and absorption of antigen.

“Desensitization” is defined as stimulation of the immune system with repeated allergen exposure in order to modify or cease the allergic response. Desensitization is considered a temporary state of clinical non-reactivity that is dependent upon persistent allergen exposure. “Tolerance induction”, on the other hand, is persistence of clinical non-reactivity to allergens when therapy is discontinued.

One object of the present invention is a composition or a set of compositions for use in a method for desensitizing and/or inducing tolerance to an allergen in an allergic patient, for treating allergy in an allergic patient or for the prophylaxis of allergy in a patient, characterized in that said composition or set of compositions comprises a dose of 5 ng to 500 μg of a local vasoconstrictor per administration event, a therapeutic dose of 0.1 ng to 10 mg of the allergen per administration event and further characterized in that the dose of the local vasoconstrictor is administered by intradermal administration to the patient and in that the dose of the allergen is administered by intradermal administration to the patient, simultaneously with or subsequently to the intradermal administration of the local vasoconstrictor, wherein the intradermal administration of the allergen is performed in the area of the dermis wherein the local vasoconstrictor is administered.

In a preferred aspect, the method for the prophylaxis of allergy in a patient is applied to a patient which is at risk at developing allergy to the administered allergen, such as a patient born from allergic parents, a patient with allergic siblings, or patients who previously demonstrated allergy to other allergens.

The allergen and the vasoconstrictor can be administered at the same time, in one single composition. Alternatively, the vasoconstrictor can be administered, in the form of a first composition, and the allergen administered subsequently in a second composition. In such case the vasoconstrictor is preferably administered between 5 and 120 minutes, more preferable about 30 minutes, before the administration of the allergen. Any of the allergen and the vasoconstrictor can be administered by intradermal injection or by other means of intradermal administration, provided that they are conveyed into the dermis of the patient. The allergen is however preferably administered by intradermal injection.

It is essential that the allergen and the vasoconstrictor be administered in the same area of the dermis, such as to achieve narrowing of blood vessels, and thus to reduce blood flow to the skin and absorption of antigen, at the point of administration of the allergen.

In a preferred aspect the invention relates to a set of compositions wherein several compositions comprise the allergen, and wherein such compositions comprising the allergen are provided with different doses of the allergen. In such case, one of said compositions comprising the allergen is administered at each administration event, the compositions being administered such as to increase the allergen dose from the first administration event to the last administration event. This is advantageous, because progressively increasing the dose of the allergen is beneficial to efficiency of the desensitization or tolerance induction to the allergen and/or to the treatment or prevention of the allergy and contributes to avoidance of adverse reactions, such as systemic reactions.

In one aspect, each of the compositions comprising the allergen also comprises the vasoconstrictor. Alternatively, the compositions comprising the allergen do not comprise the vasoconstrictor and the set of compositions further comprises at least one composition comprising the vasoconstrictor, wherein a dose of the vasoconstrictor, such as described herein, is administered before administration of each of the compositions comprising the allergen.

In a preferred aspect, at least one pharmaceutical excipient is present in the composition or in the set of compositions and is administered with the allergen. Preferably, when the invention is directed to a set of compositions, each composition comprises at least one pharmaceutical excipient, wherein the first composition comprises the vasoconstrictor with at least one suitable pharmaceutical excipient and the second composition comprises the allergen, with at least one suitable pharmaceutical excipient.

In a preferred aspect, an adjuvant is further provided to the patient by intradermal administration to potentiate the efficacy of the immunotherapy. The adjuvant can be administered alone, together with the allergen and/or together with the vasoconstrictor. Preferably, it is administered before administration of the allergen, in a way as described above, more preferably by application on the skin. More preferably, it is administered together with the vasoconstrictor before administration of the allergen, more preferably by application on the skin.

In another preferred aspect, a local anaesthetic is provided to the patient by intradermal administration. As for the adjuvant, it can be administered alone, with the allergen and/or with the vasoconstrictor. Preferably, it is administered before administration of the allergen. More preferably it is administered with the vasoconstrictor before administration of the allergen. In an even more preferred aspect, the local anaesthetic is administered together with the vasoconstrictor, even more preferably with the vasoconstrictor and the adjuvant, before administration of the allergen, and most preferably by application on the skin.

In an embodiment the adjuvant and/or the anaesthetic are administered by application on the skin between 5 and 120 minutes, preferably about 30 minutes, before the allergen and vasoconstrictor are administered simultaneously by intradermal injection.

Thus, in a particularly preferred embodiment of the invention, the vasoconstrictor, the adjuvant and the anaesthetic are all administered by application on the skin between 5 and 120 minutes, preferably about 30 minutes, before the allergen is administered by intradermal administration. This is beneficial because the vasoconstrictor and the adjuvant have sufficient time to reach optimal efficacy before the allergen is administered. Further, the anaesthetic reduces the pain caused by the injection, in cases where the allergen is administered by intradermal injection. For this effect, it is particularly preferred that the anaesthetic is administered by application to the skin, such as to reduce the pain associated with the puncture of the skin.

In a most preferred embodiment of the invention, the vasoconstrictor, the adjuvant and the anaesthetic are all administered by application on the skin between 5 and 120 minutes, preferably about 30 minutes, before the allergen is administered by intradermal injection.

According to one embodiment, the allergen is selected from the group comprising or consisting of natural allergens, modified natural allergens, synthetic allergens, recombinant allergens, allergoids, peptides or peptide fragment allergens and mixtures or combinations thereof. In a preferred aspect, the allergen is a crude allergen. Such crude allergen may be in any form suitable for administration by intradermal administration, preferably by intradermal injection, such as for example in the form of a powder or flour of the allergen suspended in liquid or in a gel.

Preferably, the allergen is obtained from or originated from plant pollen; dust; animal dander; house dust mites; fungal spores; food; venoms selected from the group of ants, bees or wasps venoms; or contact allergens; mixtures or synthetic analogues thereof.

According to a preferred embodiment of the invention, the allergen is a food allergen or an animal dander allergen selected from dogs, cats, mice, birds or rodents.

Preferably, the food allergen is selected from the group comprising milk, egg, peanuts, nuts, sesame, soy, seafood, grains, fruits or vegetables allergens.

Respiratory allergens may be derived from any of the following plants: trees, grasses, weeds. In particular, examples of tree allergens include, for example, alder, elm, olive, ash, hazel, pine, beech, heath, plane, birch, hickory, poplar, chestnut, hornbeam, lime, linden, maple, tea, cypress, myrtle, wattle, Japanese cedar, mulberry, walnut, Western red cedar, oak, willow, and any mixtures of these.

Examples of grass pollen allergens include pollen allergens from maize, Timothy grass, meadow grass, Bermuda grass, bluegrass, brome, paspalum, orchard grass, perennial rye, sweet vernal, meadow fescue, velvet, wild oat, perennial rye, common reed, June (Kentucky blue), red top, Johnson, cultivated rye, cultivated oat, cultivated wheat, meadow foxtail, Bahia, wild rye, Canary grass, couch, Sudan grass, salt grass, and any mixture thereof.

Examples of weed pollen allergens are allergens from common ragweed, western ragweed, giant ragweed, false ragweed, wormwood, oxeye daisy, Russian thistle, golden rod, mugwort, pellitory, nettles, plantain, duck weed, fat hen, sorrel, pigweed, goosefoot, dandelion, goldenrod, helianthus, sage, cocklebur, clover, alfalfa, rabbitbush, careless weed, saltbush, poverty weed, rough pigweed, yellow dock, dog fennel, and any mixtures of these. Optionally, the allergens may represent mixtures of grass-, weed-, and/or tree pollen allergens.

Respiratory allergens may be derived from any of the following fungi: Penicillium, Cladosporium, Aspergillus, Mucor, Candida, Alternaria.

Respiratory allergens may also be derived from any of the following animals: animal hair and animal dander derived from cats, dogs, birds, rodents (including hamsters, gerbils, mice, Guinea pigs, rabbits), dust mites, coackroaches or any mixture thereof.

Drug allergens include but are not limited to the following classes: antibiotics, non-steroidal anti-inflammatory drugs (NSAIDS), radio-contrast material, neuromuscular blocking agents, anti-epileptics.

Insect venom allergens may be derived from any of the following insects: honeybee, yellow jacket, wasp, paper wasp, yellow hornet, horse fly, red ant, fire ant, mosquito, black fly, deer fly, or any mixture thereof.

Contact allergens may be derived from any of the following allergens: metals, preservatives, fragrances, topical medications, P-phenylenediamine, rubber accelerators, adhesives, acrylates, fabric dyes and finishes.

According to another embodiment, the therapeutic dose is comprised between 0.1 ng and 10 mg of allergen per administration event. A “therapeutic dose” refers to the amount or quantity of any substance required to produce the desired outcome.

In accordance with the invention, the local vasoconstrictor is selected from the group comprising or consisting of epinephrine, norepinephrine, phenylephrine, levonordefrin, neuropeptide Y, peptide YY, dopamine, dobutamine, endothelin-1, serotonin, thromboxane A 2, naphazoline, ephedrine, desoxyephedrine, xylometazoline, oxymetazoline, tetrahydrozoline, phenylpropanolamine, phenylpropylmethylamine, metaraminol, norpholedrine, isoproterenol, cyclopentamine, oenethyl and mixtures thereof.

In one specific embodiment, the local vasoconstrictive agent or vasoconstrictor is a catecholamine. Catecholamines included neurotransmitters such as dopamine, epinephrine, and norepinephrine which are secreted by the adrenal glands during stress response. They are potent cutaneous vasoconstrictors and used in routine practice during administration of local anaesthetics by anaesthesiologists or dentists to decrease systemic absorption and toxicity of local anaesthetics. Interestingly, epinephrine has been shown to increase lymphatic contractions after administration (5), a mechanism, which could potentially increase flow of allergens to lymphatics when combined with allergens.

According to one embodiment, the dose of said local vasoconstrictor is between 5 ng and 500 μg per administration event. Preferably, the local vasoconstrictor is epinephrine at a dose of 5 μg per administration event.

To lower the pain of intradermal injection, a local anaesthetic may be injected intradermally at the same time as the described intradermal immunotherapeutic composition. Preferably the local anaesthetic is selected from the amino amides group consisting of benzocaine, chloroprocaine, cocaine, procaine, proparacaine, and tetracaine or from the amino esters group consisting of articaine, bupivacaine, levobupivacaine, dibucaine, etidocaine, mepivacaine, prilocaine, ropivacaine, and lidocaine.

According to one embodiment, the dose of said local anaesthetic is between 0.05 mg to 10 mg per administration event.

In another embodiment, the local anaesthetic is selected from Lidocaine, preferably Lidocaine 4%, Epinephrine, preferably Epinephrine 0.05%, or Tetracaine, preferably Tetracaine 0.5 (LET).

In an embodiment, the adjuvant is preferably selected from the group comprising aluminium salts, liposomes, toll-like receptor agonist.

Adjuvants have been used for nearly a century to increase the effectiveness of vaccination. An “adjuvant” may be defined as a substance used in combination with a specific antigen to enable a stronger immune response than when administering the antigen alone. They are commonly used in allergen-specific immunotherapy and have been shown to reduce symptoms, increase clinical efficacy of allergy treatment, and reduce the number of doses required to achieve maintenance.

The adjuvant preferably comprises any form of aluminium, oil emulsion, TLR agonist, enterotoxin, polysaccharide, and glycolipid. In a preferred embodiment, one of the following adjuvants may be used: aluminium hydroxide, delta-inulin, monophosphoryl-lipid-A, CpG, glucopyranosyl lipid adjuvant, butyrate.

As defined above, the compositions can comprise at least one suitable pharmaceutical excipient including or comprising solvents, cosolvents, diluents, surfactants, co-surfactants, thickeners, film-forming agents, stabilisers, antioxidants, solubilisers, pH-adjusting agents, colouring agents or mixtures thereof.

According to another embodiment, the composition or the set of compositions of the invention are for use in the treatment or prophylaxis of allergic diseases. Types of allergic diseases to be treated or prevented include for example allergic rhinitis, allergic conjunctivitis, allergic asthma, food allergy, drug allergy, insect allergy, contact dermatitis, atopic dermatitis.

Also described is the administration of the composition or the set of compositions via epicutaneous, subcutaneous, or intralymphatic routes. Of particular interest is the intralymphatic route, which has been shown to be a highly effective route for AIT, although associated with a high rate of systemic adverse reactions, which has limited its widespread use. Intralymphatic administration of immunotherapeutic composition may reduce the rate of systemic side effects associated with this route.

Thus, according to one embodiment, the intradermal immunotherapeutic composition of the invention for use in the treatment or prophylaxis of allergic diseases is also suitable for administration via the intralymphatic or epicutaneous route.

Also described is an intralymphatic or epicutaneous desensitization and/or tolerance induction immunotherapeutic composition suitable for injection to an allergic patient, said immunotherapeutic composition comprising per administration event: a therapeutic dose of 0.1 ng to 10 mg of allergen, between 5 ng to 500 μg of a local vasoconstrictor, and optionally at least one suitable pharmaceutical excipient, said doses being defined by administration event.

The allergen, the vasoconstrictor and suitable pharmaceutical excipient are as described above. Preferably, the immunotherapeutic composition further comprises a local anaesthetic as described above.

It is another object of the invention to provide a kit for use in a method of desensitizing and/or inducing tolerance to an allergen in an allergic patient, for treating allergy in a patient or for the prophylaxis of allergy in a patient, wherein said kit comprises:
1) a first set of vials with progressive 5 to 10-fold dilutions of a stock allergen, optionally in a suitable pharmaceutical excipient,
2) a second pre-prepared vial with a fixed concentration of local vasoconstrictor, optionally in a suitable pharmaceutical excipient, and optionally a fixed concentration of a local anaesthetic and/or of an adjuvant,
3) a third set of vials for mixing said first vial with said second vial so as to prepare a dose of said allergen,
   characterized in that said dose of said allergen prepared in step 3) is administered by intradermal administration and wherein said dose of said allergen comprises from 0.1 ng to 10 mg of said allergen and from 5 ng to 500 μg of said local vasoconstrictor per administration event.

Preferably said dose of said allergen prepared in step 3) is administered by intradermal injection.

The “stock allergen” is defined as the most concentrated allergen (for example: 1/10 w:v). Preferably the stock allergen is a stock allergenic extract.

Preparation of progressive dilutions can be performed using a syringe and needle by for example taking 1 mL of stock allergen and optionally diluting in 9 mL of suitable excipient for a 10-fold dilution or taking 1 mL of allergen and optionally diluting in 4 mL of suitable excipient for a 5-fold dilution. An example of excipient could be normal saline or human serum albumin.

A further object of the invention is to provide an intradermal desensitization and/or tolerance induction immunotherapeutic kit, comprising:
a set of vials with 5 to 10-fold dilutions from a stock allergen containing progressively lower concentrations of allergen and a fixed dose of local vasoconstrictor, optionally in a suitable pharmaceutical excipient.

In a preferred aspect, the second pre-prepared vial does not comprise a local anaesthetic and/or an adjuvant and the kit further comprises a separate composition comprising a local anaesthetic and/or an adjuvant, said composition being suitable for intradermal administration of said anaesthetic and/or adjuvant, and wherein said anaesthetic and/or adjuvant is administered by intradermal administration before administration of the said dose of said allergen prepared in step 3).

More preferably, such separate composition comprising a local anaesthetic and/or an adjuvant is administered by application on the skin between 5 and 120 minutes, preferably about 30 minutes, before the allergen is administered by intradermal administration, preferably by intradermal injection.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

Sterile fish extract ( 1/50 weight volume) was diluted 10, 100, 1000, 10,000, 100,000, and 1 million-fold in separate vials. Skin prick tests with these different concentrations were performed on the volar forearm of a fish-allergic subject. The lowest concentration yielding a positive skin prick test (LCP) (defined as 3 mm above negative control after 15 minutes) was determined.

An intradermal test with 0.05 mL of the LCP was performed on the volar forearm. Intradermal tests were performed every 20 minutes up to the highest concentration yielding a 3 mm increase compared to initial wheal and/or erythema with a sum of the longest diameter+orthogonal diameter of more than 100 mm. This was considered the starting dose for intradermal immunotherapy. The patient received intradermal injections of fish extract weekly to biweekly for a duration of 5 months, followed by a pause of 5 months. The patient was then treated with allergenic extract with epinephrine (final concentration of 0.1 mg/mL) for an additional period of 1 month. During the treatment period, intradermal allergenic extract dose was adapted depending on size of local reactions.

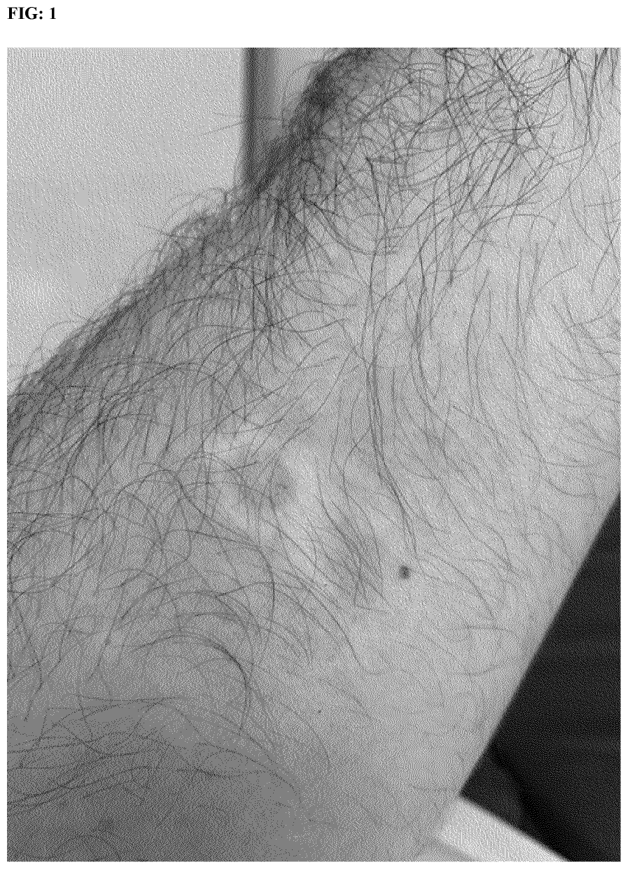

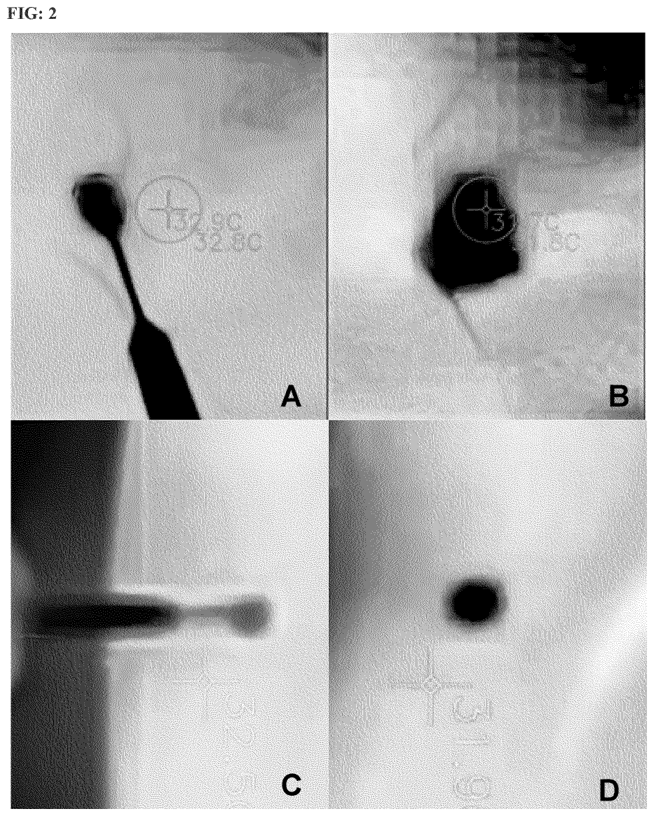

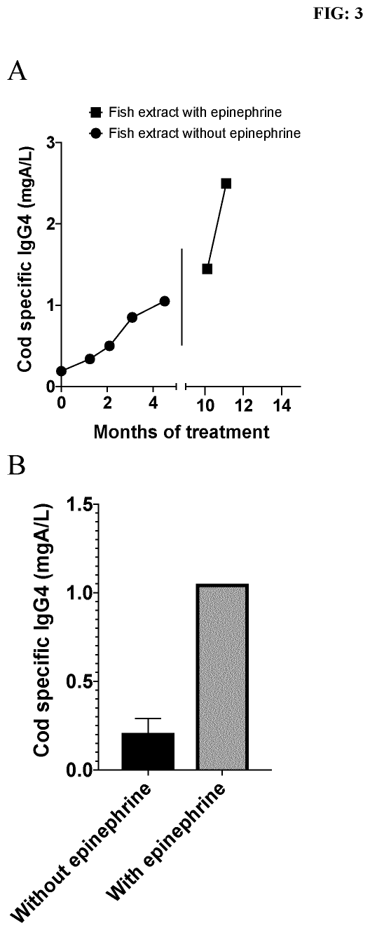

As shown in FIG. 1, intradermal injection of fish extract with epinephrine causes skin blanching consistent with local vasoconstriction. This is equally showed by thermography, which indicates rapid uptake of allergenic extract without epinephrine by local cutaneous blood vessels after intradermal injection (FIGS. 2A and 2B). This effect is abrogated by the addition of epinephrine to the intradermal extract (FIGS. 2C and 2D). Treatment with cod allergenic extract increased IgG4 levels, which are associated with tolerance to ingested allergen and increase during other forms of AIT. Cod-specific IgG4 levels increased slowly in the first 5 months of treatment with cod allergic extract without epinephrine (absolute increase of 0.86 mgA/L) (FIG. 3A). Pursuing treatment with cod extract in the presence of epinephrine (100 mcg/mL) caused a much faster increase in cod-specific IgG4 after only 1 month of treatment (absolute increase of 1.05 mgA/L) (FIG. 3A). Monthly cod specific IgG4 increase was significantly lower using cod extract without epinephrine (mean of 0.21 mgA/L per month) when compared to cod extract with epinephrine (1.05 mgA/L in one month) (FIG. 3B). This is consistent with epinephrine having an adjuvant effect.

### LIST OF REFERENCES

1. Cox L, Nelson H, Lockey R, Calabria C, Chacko T, Finegold I, et al. Allergen immunotherapy: a practice parameter third update. J Allergy Clin Immunol. 2011; 127 (1 Suppl): S1-55.
2. Senti G, Kundig T M. Novel Delivery Routes for Allergy Immunotherapy: Intralymphatic, Epicutaneous, and Intradermal. Immunol Allergy Clin North Am. 2016; 36(1):25-37.
3. Burks A W, Sampson H A, Plaut M, Lack G, Akdis C A. Treatment for food allergy. J Allergy Clin Immunol. 2018; 141(1):1-9.

4. Nelson H S, Lahr J, Rule R, Bock A, Leung D. Treatment of anaphylactic sensitivity to peanuts by immunotherapy with injections of aqueous peanut extract. J Allergy Clin Immunol. 1997; 99 (6 Pt 1):744-51.
5. Kwon S, Sevick-Muraca E M. Effect of lidocaine with and without epinephrine on lymphatic contractile activity in mice in vivo. J Anesth. 2016; 30(6):1091-4.
6. Ghali S, Knox K R, Verbesey J, Scarpidis U, Izadi K, Ganchi P A. Effects of lidocaine and epinephrine on cutaneous blood flow. J Plast Reconstr Aesthet Surg. 2008; 61(10):1226-31.
7. Cederholm I, Evers H, Lofstrom J B. Skin blood flow after intradermal injection of ropivacaine in various concentrations with and without epinephrine evaluated by laser Doppler flowmetry. Reg Anesth. 1992; 17(6):322-8.

8. Sapsaprang S, Boonard K, Pacharn P, Srisuwatchari W, Visitsunthorn N, Jirapongsananuruk O. Epinephrine-coated syringe for SCIT reduced local reactions: A randomized, double-blind, placebo-controlled trial. J Allergy Clin Immunol Pract. 2020; 8(4):1465-7 el.

9. Mustafa S S, Vadamalai K, Bingemann T, Ramsey A. Efficacy of epinephrine and diphenhydramine rinses in decreasing local reactions to subcutaneous aeroallergen immunotherapy. Allergy Asthma Proc. 2020; 41(1):52-8.
10. Kersey T W, Van Eyk J, Lannin D R, Chua A N, Tafra L. Comparison of intradermal and subcutaneous injections in lymphatic mapping. J Surg Res. 2001; 96(2):255-9.
11. Phillips E W, Phoenix M D Ariz, Intradermal pollen therapy during the attack, Read at the Eleventh Annual Meeting of the Association for the Study of Allergy, Milwaukee, Wis., Jun. 12 and 13 1933.
12. David Ordman, Allergic desensitization by the intradermal route. South African Medical Journal, 1961; 35(50):617-620.
13. Duke W. W., New method of administering pollen extract for purpose of preventing reactions: A subcuticular method, Journal of the American Medical Association, 1930; 94(11):767-771.

1. A method for desensitizing and/or inducing tolerance to an allergen in an allergic patient, for treating allergy in an allergic patient or for the prophylaxis of allergy in a patient, the method comprising administering a composition or a set of compositions to the patient, wherein the composition or set of compositions comprises a dose of 5 ng to 500 μg per administration event of a local vasoconstrictor and a therapeutic dose of 0.1 ng to 10 mg per administration event of allergen;
wherein the dose of the local vasoconstrictor is administered by intradermal administration to the patient;
wherein the therapeutic dose of the allergen is administered by intradermal administration to the patient, simultaneously with or subsequently to the intradermal administration of the local vasoconstrictor; and
wherein the intradermal administration of the allergen is performed in the area of the dermis where the local vasoconstrictor is administered.

2. The method according to claim 1, wherein said composition comprises, per administration event: a mixture of a therapeutic dose of 0.1 ng to 10 mg of allergen and between 5 ng to 500 μg of a local vasoconstrictor.

3. The method according to claim 2, wherein said composition is administered by intradermal injection and comprises, per injection: a mixture of a therapeutic dose of 0.1 ng to 10 mg of allergen and between 5 ng to 500 μg of a local vasoconstrictor.

4. The method according to claim 1, wherein the set of compositions is administered to the patient and the set of compositions comprises a first composition suitable for application on the skin and a second composition which is injectable;
wherein the first composition comprises the local vasoconstrictor and is administered to the patient by application on the skin; and
wherein the second composition comprises the allergen and is administered to the patient after administration of the first composition by intradermal injection in the area of the skin where the first composition was applied.

5. The method according to claim 4, wherein the first composition is in the form of a cream, a paste, a gel, an ointment, a solution, a lotion or an oil, optionally administered in the form of an impregnated patch.

6. The method according to claim 4, wherein the second composition comprises a crude allergen and is provided in the form of a solution or in the form of a powder or flour of the allergen suspended in liquid or in a gel.

7. The method according to claim 4, wherein the second composition is administered between 5 and 120 minutes after administration of the first composition.

8. The method according to claim 1, wherein the method further comprises intradermal administration of an adjuvant prior to or simultaneously with the administration of the allergen.

9. The method according to claim 8, wherein the adjuvant is administered together with the local vasoconstrictor.

10. The method according to claim 1, wherein the method further comprises administration of a local anaesthetic at a dose of 0.05 mg to 10 mg, before or simultaneously to the administration of the allergen.

11. The method according to claim 10, wherein the local anaesthetic is provided together with the local vasoconstrictor.

12. The method according to claim 1, wherein the allergen is a natural allergen, a modified natural allergen, a synthetic allergen, a recombinant allergen, an allergoid, a peptides or peptide fragment allergen or a mixtures or combinations thereof.

13. The method according to claim 1, wherein the allergen is originated from plant pollen; dust; animal dander; house dust mites; fungal spores; food; a venoms selected from the group of ant, bee, and wasp venom; a mixture or synthetic analogue thereof.

14. The method according to claim 1, wherein the allergen is a food allergen or an animal dander allergen selected from dogs, cats, mouse, bird or rodent.

15. The method according to claim 1, wherein the food allergen is milk, egg, peanut, nut, legume, sesame, soy, seafood, grain, fruits or vegetables allergen.

16. The method according to claim 1, wherein the local vasoconstrictor is selected from the group consisting of epinephrine, norepinephrine, phenylephrine, levonordefrin, neuropeptide Y, peptide YY, dopamine, dobutamine, endothelin-1, serotonin, thromboxane A 2, naphazoline, ephedrine, desoxyephedrine, xylometazoline, oxymetazoline, tetrahydrozoline, phenylpropanolamine, phenylpropylmethylamine, metaraminol, norpholedrine, isoproterenol, cyclopentamine, oenethyl and a mixtures thereof.

17. The method according to claim 1, wherein the adjuvant is an aluminium salt, a liposome or a toll-like receptor agonist.

18. An immunotherapeutic kit comprising:
1) a first set of vials with progressive 5 to 10-fold dilutions of a stock allergen, optionally in a suitable pharmaceutical excipient,
2) a second pre-prepared vial with a fixed concentration of local vasoconstrictor, optionally in a suitable pharmaceutical excipient and optionally a fixed concentration of a local anaesthetic and/or of an adjuvant, and
3) a third set of vials for mixing said first vial with said second vial so as to prepare a dose of said allergen,
wherein said dose of said allergen comprises from 0.1 ng to 10 mg of said allergen and from 5 ng to 500 μg of said local vasoconstrictor per administration event.

19. The immunotherapeutic kit according to claim 18, wherein the second pre-prepared vial does not comprise a local anaesthetic and/or an adjuvant, and the kit further comprises a separate composition comprising said local anaesthetic and/or adjuvant, wherein said separate composition is suitable for intradermal administration of said anaesthetic and/or adjuvant.

20. The immunotherapeutic kit according to claim 18,
wherein the local vasoconstrictor is selected from the group consisting of epinephrine, norepinephrine, phenylephrine, levonordefrin, neuropeptide Y, peptide YY, dopamine, dobutamine, endothelin-1, serotonin, thromboxane A 2, naphazoline, ephedrine, desoxyephedrine, xylometazoline, oxymetazoline, tetrahydrozoline, phenylpropanolamine, phenylpropylmethylamine, metaraminol, norpholedrine, isoproterenol, cyclopentamine, oenethyl and a mixture thereof; and/or
wherein the adjuvant is an aluminium salt, a liposome or a toll-like receptor agonist.
